(19) United States
(12) Patent Application Publication
(10) Pub. No.: US 2025/0368653 A1
(43) Pub. Date: Dec. 4, 2025

(54) SOLID FORMS COMPRISING (S)-7-(1-ACRYLOYLPIPERIDIN-4-YL)-2-(4-PHENOXYPHENYL)-4,5,6,7-TETRAHYDROPYRAZOLO[1,5-A]PYRIMIDINE-3-CARBOXAMIDE, AND OXALIC ACID, COMPOSITIONS AND METHODS OF USE THEREOF

(71) Applicant: BeiGene Switzerland GmbH, Basel (CH)

(72) Inventor: Qian Li, Beijing (CN)

(21) Appl. No.: 19/062,656

(22) Filed: Feb. 25, 2025

Related U.S. Application Data
(63) Continuation of application No. PCT/CN2023/114741, filed on Aug. 24, 2023.

(30) Foreign Application Priority Data
Aug. 25, 2022 (WO) PCT/CN2022/114859

(51) Int. Cl. C07D 487/04

(52) U.S. Cl. CPC C07D 487/04

(57) ABSTRACT

Provided herein are formulations, processes, solid forms and methods of use relating to (S)-7-(1-acryloylpiperidin-4-yl)-2-(4-phenoxyphenyl)-4,5,6,7-tetrahydropyrazolo[1,5-a]pyrimidine-3-carboxamide.

This application is a continuation of PCT/CN2023/114741 filed Aug. 24, 2023, published Feb. 29, 2024, under International Publication No. WO 2024/041614 A1, which claims priority to PCT/CN2022/114859 filed Aug. 25, 2022, the disclosures of which are incorporated herein by reference in their entireties.

### BACKGROUND OF THE INVENTION

### Field

Provided herein are solid forms comprising(S)-7-(1-acryloylpiperidin-4-yl)-2-(4-phenoxyphenyl)-4,5,6,7-tetrahydropyrazolo[1,5-a]pyrimidine-3-carboxamide and oxalic acid. Pharmaceutical compositions comprising such solid forms, and methods of use for treating, preventing, and managing various disorders are also provided herein.

### Background

The identification and selection of a solid form of a pharmaceutical compound is complex, given that a change in a solid form may affect a variety of physical and chemical properties, which may provide benefits or drawbacks in processing, formulation, stability, and bioavailability, among other important pharmaceutical characteristics. Potential pharmaceutical solids include crystalline solids and amorphous solids. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical or chemical stability (see, e.g., S. R. Vippagunta et al., Adv. Drug. Deliv. Rev., (2001) 48:3-26; L. Yu, Adv. Drug. Deliv. Rev., (2001) 48:27-42).

Whether crystalline or amorphous, potential solid forms of a pharmaceutical compound include single-component and multiple-component solids. Single-component solids consist essentially of the pharmaceutical compound in the absence of other compounds. Variety among single-component crystalline materials may potentially arise from the phenomenon of polymorphism, wherein multiple three-dimensional arrangements exist for a particular pharmaceutical compound (see, e.g., S. R. Byrn et al., Solid State Chemistry of Drugs, (1999) SSCI, West Lafayette). The importance of discovering polymorphs was underscored by the case of Ritonavir, an HIV protease inhibitor that was formulated as soft gelatin capsules. About two years after the product was launched, the unanticipated precipitation of a new, less soluble polymorph in the formulation necessitated the withdrawal of the product from the market until a more consistent formulation could be developed (see S. R. Chemburkar et al., Org. Process Res. Dev., (2000) 4:413-417).

Additional diversity among the potential solid forms of a pharmaceutical compound may arise from the possibility of multiple-component solids. Crystalline solids comprising two or more ionic species are termed salts (see, e.g., Handbook of Pharmaceutical Salts: Properties, Selection and Use, P. H. Stahl and C. G. Wermuth, Eds., (2002), Wiley, Weinheim). Additional types of multiple-component solids that may potentially offer other property improvements for a pharmaceutical compound or salt thereof include, e.g., hydrates, solvates, cocrystals and clathrates, among others (see, e.g., S. R. Byrn et al., Solid State Chemistry of Drugs, (1999) SSCI, West Lafayette). Moreover, multiple-component crystal forms may potentially be susceptible to polymorphism, wherein a given multiple-component composition may exist in more than one three-dimensional crystalline arrangement. The discovery of solid forms is of great importance in the development of a safe, effective, stable and marketable pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.: 3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

Cocrystals are crystalline molecular complexes of two or more non-volatile compounds bound together in a crystal lattice by non-ionic interactions. Pharmaceutical cocrystals are cocrystals of a therapeutic compound, e.g., an active pharmaceutical ingredient (API), and one or more non-volatile compound(s) (referred to herein as coformer). A coformer in a pharmaceutical cocrystal is typically selected from non-toxic pharmaceutically acceptable molecules, such as, for example, food additives, preservatives, pharmaceutical excipients, or other APIs. In recent years, pharmaceutical cocrystals have emerged as a possible alternative approach to enhance physicochemical properties of drug products. The variety of possible solid forms creates potential diversity in physical and chemical properties for a given pharmaceutical compound.

The compound chemically named(S)-7-(1-acryloylpiperidin-4-yl)-2-(4-phenoxyphenyl)-4,5,6,7-tetrahydropyrazolo[1,5-a]pyrimidine-3-carboxamide and tautomers thereof (collectively referred to herein as “Compound 1”) are disclosed in U.S. Pat. No. 9,447,106, issued on Sep. 20, 2016, and International Pub. No. WO2014/173289, the entireties of each of which are incorporated by reference herein.

Crystalline Form A of Compound 1 is disclosed in U.S. Pat. No. 10,927,117, issued on Feb. 23, 2021, and International Pub. No. WO2018/033853, the entireties of each of which are incorporated by reference herein.

Citation or identification of any reference in Section 2 of this application is not to be construed as an admission that the reference is prior art to the present application.

### SUMMARY

Provided herein are solid forms comprising Compound 1:

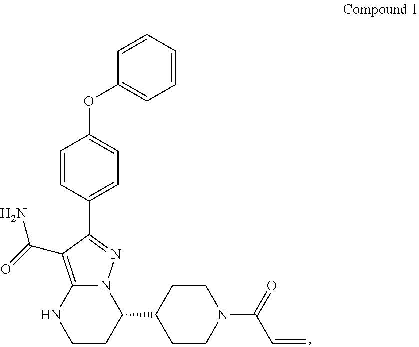 [SMILES: C=CC(=O)N1CCC([C@@H]2CCNc3c(C(N)=O)c(-c4ccc(Oc5ccccc5)cc4)nn32)CC1]

having the name(S)-7-(1-acryloylpiperidin-4-yl)-2-(4-phenoxyphenyl)-4,5,6,7-tetrahydropyrazolo[1,5-a]pyrimidine-3-carboxamide, including tautomers thereof, and oxalic acid. Also provided are methods of preparing, isolating, and characterizing the solid forms.

Further provided herein are crystal forms comprising Compound 1. One crystal form provided herein has an X-ray powder diffraction pattern comprising characteristic X-ray powder diffraction peaks at a two-theta angle of approximately 8.17, 11.65 or 21.61 degrees. In one embodiment, the X-ray powder diffraction pattern can further comprise characteristic X-ray powder diffraction peaks at a two-theta angle of approximately 8.56, 18.54, or 19.56 degrees.

In one embodiment, the solid form is a cocrystal. In one embodiment, the solid form is a salt.

In one embodiment, the crystal form has a thermogravimetric analysis thermogram comprising a total mass loss of approximately 0.7% of the total mass of the crystal form when heated from about 17.6° C. to about 130° C. In one embodiment, the crystal form has a differential scanning calorimetry thermogram comprising an endothermic event with a maximum at approximately 160.4° C. when heated from about 50° C. to about 200° C. In one embodiment, the crystal form has a differential scanning calorimetry thermogram comprising an endothermic event with an onset temperature at approximately 159.1° C. when heated from about 50° C. to about 200° C.

In one embodiment, the molar ratio of oxalic acid to Compound 1 in the crystal form is about 0.5. The crystal form can be substantially pure.

All the solid forms and the pharmaceutical compositions provided herein can be used as a medicament. In certain embodiments, solid forms of Compound 1 are useful for treating or preventing cancer and conditions treatable or preventable by inhibition of a kinase pathway, for example, the BTK pathway. All the solid form and the pharmaceutical composition can be used in methods for treating or preventing a cancer, an allergic disease, an autoimmune disease, an inflammatory disease, a combination of two or more thereof, or a condition treatable or preventable by inhibition of a kinase pathway. The methods comprise administering an effective amount of a solid form or a pharmaceutical composition to a subject in need thereof. The kinase pathway is the BTK kinase pathway. In one embodiment, provided herein is a method of treating a B-cell proliferative disease, selected from chronic lymphocytic lymphoma, non-Hodgkin's lymphoma, diffuse large B cell lymphoma, mantle cell lymphoma, follicular lymphoma, chronic lymphocytic leukemia, or a combination of two or more thereof, in a subject by administering to the subject a solid form disclosed herein.

All the solid forms and the pharmaceutical compositions provided herein can be used in methods for achieving a Response Evaluation Criteria in Solid Tumors (RECIST 1.1) of complete response, partial response or stable disease in a subject. The methods comprise administering an effective amount of a solid form or a pharmaceutical composition to a subject having a solid tumor.

All the solid forms and the pharmaceutical compositions provided herein can be used in methods for improving International Workshop Criteria (IWC) for NHL, or Eastern Cooperative Oncology Group Performance Status (ECOG). The methods comprise administering an effective amount of a solid form or a pharmaceutical composition to a subject in need thereof.

The present embodiments can be understood more fully by reference to the detailed description and examples, which are intended to exemplify non-limiting embodiments.

### BRIEF DESCRIPTION OF THE DRAWINGS

FIG. 1 depicts an X-ray powder diffractogram pattern of Form 1.

FIG. 2 depicts a thermogravimetric analysis of Form 1.

FIG. 3 depicts a differential scanning calorimetry analysis of Form 1.

FIG. 4 depicts a 1H NMR of Form 1.

FIG. 5 depicts a DVS result showing that water uptake of Form 1 at 25° C./80% RH was lower than 0.2%, indicating it was non hygroscopic.

FIG. 6 depicts a solutability profile of Form 1, and Form A of Compound 1, and amorphous form of Compound 1 at room temperature.

FIG. 7 depicts a solutability profile of Form 1, and Form A of Compound 1, and amorphous form of Compound 1 at 37° C.

### DETAILED DESCRIPTION

### Definitions

As used herein, and unless otherwise specified, the terms “oxalic acid” and “oxalate” refer to both non-ionized/unassociated oxalic acid and ionized/associated oxalic acid.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, and solubility studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2 degrees two theta while still describing the particular XRPD peak.

As used herein, and unless otherwise specified, a crystalline that is “pure,” i.e., substantially free of other crystalline or amorphous forms, contains less than about 10% by weight of one or more other crystalline or amorphous forms, less than about 5% by weight of one or more other crystalline or amorphous forms, less than about 3% by weight of one or more other crystalline or amorphous forms, or less than about 1% by weight of one or more other crystalline or amorphous forms.

As used herein and unless otherwise indicated, the term “substantially pure” when used to describe a polymorph of a compound, i.e. a crystal form or an amorphous form of a compound, means a crystal form or an amorphous form of the compound that comprises that crystal form or amorphous form and is substantially free of other polymorphs of the compound. A substantially pure crystal form is no less than about 95% pure, no less than about 96% pure, no less than about 97% pure, no less than about 98% pure, no less than about 98.5% pure, no less than about 99% pure, no less than about 99.5% pure, or no less than about 99.8% pure. In certain embodiments, a form that is substantially pure contains less than about 10%, 9%, 8%, 7%, 6%, 5%, 4%, 3%, 2%, 1%, 0.5%, 0.4%, 0.3%, 0.2%, 0.1%, 0.05%, or 0.01% of one or more other polymorphs on a weight basis.

As used herein, and unless otherwise specified, a solid form that is “substantially physically pure” is substantially free from other solid forms. In certain embodiments, a crystal form that is substantially physically pure contains less than about 10%, 9%, 8%, 7%, 6%, 5%, 4%, 3%, 2%, 1%, 0.5%, 0.4%, 0.3%, 0.2%, 0.1%, 0.05%, or 0.01% of one or more other solid forms on a weight basis. The detection of other solid forms can be accomplished by any method apparent to a person of ordinary skill in the art, including, but not limited to, diffraction analysis, thermal analysis, elemental combustion analysis and/or spectroscopic analysis.

As used herein, and unless otherwise specified, a solid form that is “substantially chemically pure” is substantially free from other chemical compounds (i.e., chemical impurities). In certain embodiments, a solid form that is substantially chemically pure contains less than about 10%, 9%, 8%, 7%, 6%, 5%, 4%, 3%, 2%, 1%, 0.5%, 0.4%, 0.3%, 0.2%, 0.1%, 0.05%, or 0.01% of one or more other chemical compounds on a weight basis. The detection of other chemical compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

As used herein, and unless otherwise indicated, a chemical compound, solid form, or composition that is “substantially free” of another chemical compound, solid form, or composition means that the compound, solid form, or composition contains, in certain embodiments, less than about 10%, 9%, 8%, 7%, 6%, 5%, 4%, 3%, 2%, 1%, 0.5%, 0.4%, 0.3%, 0.2% 0.1%, 0.05%, or 0.01% by weight of the other compound, solid form, or composition.

Unless otherwise specified, the terms “solvate” and “solvated,” as used herein, refer to a solid form of a substance which contains solvent. The terms “hydrate” and “hydrated” refer to a solvate wherein the solvent is water. “Polymorphs of solvates” refer to the existence of more than one solid form for a particular solvate composition. Similarly, “polymorphs of hydrates” refer to the existence of more than one solid form for a particular hydrate composition. The term “desolvated solvate,” as used herein, refers to a solid form of a substance which can be made by removing the solvent from a solvate. The terms “solvate” and “solvated,” as used herein, can also refer to a solvate of a salt, cocrystal, or molecular complex. The terms “hydrate” and “hydrated,” as used herein, can also refer to a hydrate of a salt, cocrystal, or molecular complex.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

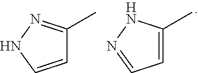

As readily understood by one skilled in the art, a wide variety of functional groups and other structures may exhibit tautomerism and all tautomers of Compound 1 are within the scope of the present invention.

Unless otherwise specified, the term “composition” as used herein is intended to encompass a product comprising the specified ingredient(s) (and in the specified amount(s), if indicated), as well as any product which results, directly or indirectly, from combination of the specified ingredient(s) in the specified amount(s). By “pharmaceutically acceptable,” it is meant a diluent, excipient, or carrier in a formulation must be compatible with the other ingredient(s) of the formulation and not deleterious to the recipient thereof.

The term “solid form” refers to a physical form which is not predominantly in a liquid or a gaseous state. As used herein and unless otherwise specified, the term “solid form,” when used herein to refer to Compound 1, refers to a physical form comprising Compound 1 which is not predominantly in a liquid or a gaseous state. A solid form may be a crystalline form or a mixture thereof. In certain embodiments, a solid form may be a liquid crystal. In certain embodiments, the term “solid forms comprising Compound 1” includes crystal forms comprising Compound 1. In one embodiment, the solid form of Compound 1 is Form 1.

As used herein and unless otherwise specified, the term “crystalline” when used to describe a compound, substance, modification, material, component, or product, unless otherwise specified, means that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, MD (2005); The United States Pharmacopeia, 23^{rd} ed., 1843-1844 (1995).

The term “crystal form” or “crystalline form” refers to a solid form that is crystalline. In certain embodiments, crystal forms include salts. In certain embodiments, a crystal form of a substance may be substantially free of amorphous forms and/or other crystal forms. In certain embodiments, a crystal form of a substance may contain less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, or less than about 50% by weight of one or more amorphous forms and/or other crystal forms. In certain embodiments, a crystal form of a substance may be physically and/or chemically pure. In certain embodiments, a crystal form of a substance may be about 99%, about 98%, about 97%, about 96%, about 95%, about 94%, about 93%, about 92%, about 91%, or about 90% physically and/or chemically pure.

Unless otherwise specified, the terms “polymorph,” “polymorphic form,” “polymorphs,” “polymorphic forms,” and related terms herein refer to two or more crystal forms that consist essentially of the same molecule, molecules or ions. Different polymorphs may have different physical properties, such as, for example, melting temperatures, heats of fusion, solubilities, dissolution rates, and/or vibrational spectra as a result of a different arrangement or conformation of the molecules or ions in the crystal lattice. The differences in physical properties exhibited by polymorphs may affect pharmaceutical parameters, such as storage stability, compressibility and density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically a more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing; for example, one polymorph might be more likely to form solvates or might be difficult to filter and wash free of impurities (e.g., particle shape and size distribution might be different between polymorphs).

Unless otherwise specified, the term “cocrystal” as used herein, refers to a crystalline material comprised of Compound 1, including tautomers thereof, and one or more non-volative compounds bound together in a crystal lattice by non-covalent interactions.

Unless otherwise specified, the term “amorphous” or “amorphous form” means that the substance, component, or product in question is not substantially crystalline as determined by X-ray diffraction. In particular, the term “amorphous form” describes a disordered solid form, i.e., a solid form lacking long range crystalline order. In certain embodiments, an amorphous form of a substance may be substantially free of other amorphous forms and/or crystal forms. In certain embodiments, an amorphous form of a substance may contain less than about 1%, less than about 2%, less than about 3%, less than about 4%, less than about 5%, less than about 10%, less than about 15%, less than about 20%, less than about 25%, less than about 30%, less than about 35%, less than about 40%, less than about 45%, or less than about 50% by weight of one or more other amorphous forms and/or crystal forms on a weight basis. In certain embodiments, an amorphous form of a substance may be physically and/or chemically pure. In certain embodiments, an amorphous form of a substance be about 99%, about 98%, about 97%, about 96%, about 95%, about 94%, about 93%, about 92%, about 91%, or about 90% physically and/or chemically pure.

“Treating” as used herein, means an alleviation, in whole or in part, of the disease or disorder, or symptoms associated with the disease or disorder, or slowing, or halting of further progression or worsening of the disease or disorder, or symptoms associated with the disease or disorder.

“Preventing” as used herein, means prevention of the onset, recurrence, or spread of the disease or disorder, or symptoms associated with the disorder or disease, in a patient at risk for developing the disease or disorder.

The term “effective amount” in connection with a solid form of Compound 1 means, in one embodiment, an amount capable of alleviating, in whole or in part, symptoms associated with a disorder or disease, or slowing or halting further progression or worsening of those symptoms, or, in another embodiment, an amount capable of preventing or providing prophylaxis for the disease or disorder in a subject at risk for developing the disease or disorder as disclosed herein, such as cancer. In one embodiment, an effective amount of a solid form of Compound 1 is an amount that inhibits a kinase in a cell, such as, for example, in vitro or in vivo. In one embodiment the kinase is BTK. In some embodiments, the effective amount of a solid form of Compound 1 inhibits the kinase in a cell by 10%, 20%, 30%, 40%, 50%, 60%, 70%, 80%, 90% or 99%, compared to the activity of the kinase in an untreated cell. The effective amount of a solid form of Compound 1, for example in a pharmaceutical composition, may be at a level that will exercise the desired effect; for example, about 0.005 mg/kg of a subject's body weight to about 100 mg/kg of a patient's body weight in unit dosage for both oral and parenteral administration. As will be apparent to those skilled in the art, it is to be expected that the effective amount of a solid form of Compound 1 disclosed herein may vary depending on the indication being treated, e.g., the effective amount of a solid form of Compound 1 would likely be different for treating patients suffering from, or at risk for, inflammatory conditions relative to the effective amount of a solid form of Compound 1 for treating patients suffering from, or at risk of, a different disorder, e.g., cancer or a metabolic disorder.

“Patient” or “subject” is defined herein to include animals, such as mammals, including, but not limited to, primates (e.g., humans), cows, sheep, goats, horses, dogs, cats, rabbits, rats, mice, monkeys, chickens, turkeys, quails, or guinea pigs and the like. In specific embodiments, the patient or subject is a human.

The term “cancer” refers to B-cell proliferative disease, selected from chronic lymphocytic lymphoma, non-Hodgkin's lymphoma, diffuse large B cell lymphoma, mantle cell lymphoma, follicular lymphoma, chronic lymphocytic leukemia (CLL), or a combination of two or more thereof.

In certain embodiments, the treatment of lymphoma may be assessed by the International Workshop Criteria (IWC) for non-Hodgkin lymphoma (NHL) (see Cheson B D, Pfistner B, Juweid, M E, et. al. Revised Response Criteria for Malignant Lymphoma. J. Clin. Oncol: 2007: (25) 579-586), using the response and endpoint definitions shown below:

| Response | Definition | Nodal Masses | Spleen, liver | Bone Marrow |
|---|---|---|---|---|
| CR | Disappearance | (a) FDG-avid or PET | Not | Infiltrate cleared |
| | of all evidence | positive prior to therapy; | palpable, | on repeat biopsy; if |
| | of disease | mass of any size permitted | nodules | indeterminate by |
| | | if PET negative | disappeared | morphology, |
| | | (b) Variably FDG-avid or | | immunohistochemistry |
| | | PET negative; regression | | should be negative |
| | | to normal size on CT | | |
| PR | Regression of | ≥50% decrease in SPD of | ≥50% | Irrelevant if |
| | measurable | up to 6 largest dominant | decrease in | positive prior to |
| | disease and no | masses; no increase in size | SPD of | therapy; cell type |
| | new sites | of other nodes | nodules (for | should be specified |
| | | (a) FDG-avid or PET | single | |
| | | positive prior to therapy; | nodule in | |
| | | one or more PET positive | greatest | |
| | | at previously involved site | transverse | |
| | | (b) Variably FDG-avid or | diameter); | |
| | | PET negative; regression | no increase | |
| | | on CT | in size of | |
| | | | liver or | |
| | | | spleen | |
| SD | Failure to | (a) FDG-avid or PET | | |
| | attain CR/PR | positive prior to therapy; | | |
| | or PD | PET positive at prior sites | | |
| | | of disease and no new | | |
| | | sites on CT or PET | | |
| | | (b) Variably FDG-avid or | | |
| | | PET negative; no change | | |
| | | in size of previous lesions | | |
| | | on CT | | |
| PD or | Any new | Appearance of a new | ≥50% | New or recurrent |
| relapsed | lesion or | lesion(s) ≥1.5 cm in any | increase | involvement |
| disease | increase | axis, ≥50% increase in | from nadir in | |
| | by ≥50% of | SPD of more than one node, | the SPD of | |
| | previously | or ≥50% increase in | any previous | |
| | involved sites | longest diameter of a | lesions | |
| | from nadir | previously identifed | | |
| | | node ≥1 cm in short axis | | |
| | | Lesions PET positive if | | |
| | | FDG-avid lymphoma or | | |
| | | PET positive prior to | | |
| | | therapy | | |

Abbreviations: CR, complete remission; FDG, [¹⁸F]fluorodeoxyglucose; PET, positron emission tomography; CT, computed tomography; PR, partial remission; SPD, sum of the product of the diameters; SD, stable disease; PD, progressive disease.

| End point | Patients | Definition | Measured from |
|---|---|---|---|
| Primary | | | |
| Overall survival | All | Death as a result of any cause | Entry onto study |
| Progression-free | All | Disease progression or death as a result of | Entry onto study |
| survival | | any cause | |
| Secondary | | | |
| Event-free survival | All | Failure of treatment or death as result of any | Entry onto study |
| | | cause | |
| Time to | All | Time to progression or death as a result of | Entry onto study |
| progression | | lymphoma | |
| Disease-free | In CR | Time to relapse or death as a result of | Documentation |
| survival | | lymphoma or acute toxicity of treatment | of response |
| Response duration | In CR or | Time to relapse or progression | Documentation |
| | PR | | of response |
| Lymphoma- | All | Time to death as a result of lymphoma | Entry onto study |
| specific survival | | | |
| Time to next | All | Time to new treatment | End of primary |
| treatment | | | treatment |

Abbreviations: CR: complete remission; PR: partial remission.

In one embodiment, the end point for lymphoma is evidence of clinical benefit. Clinical benefit may reflect improvement in quality of life, or reduction in patient symptoms, transfusion requirements, frequent infections, or other parameters. Time to reappearance or progression of lymphoma-related symptoms can also be used in this end point.

In certain embodiments, the treatment of CLL may be assessed by the International Workshop Guidelines for CLL (see Hallek M, Cheson B D, Catovsky D, et al. Guidelines for the diagnosis and treatment of chronic lymphocytic leukemia: a report from the International Workshop on Chronic Lymphocytic Leukemia updating the National Cancer Institute-Working Group 1996 guidelines. Blood, 2008; (111) 12:5446-5456) using the response and endpoint definitions shown therein and in particular:

| Parameter | CR | PR | PD |
|---|---|---|---|
| Group A | | | |
| Lymphadenopathy^{†} | None >1.5 cm | Decrease ≥50% | Increase ≥50% |
| Hepatomegaly | None | Decrease ≥50% | Increase ≥50% |
| Splenomegaly | None | Decrease ≥50% | Increase ≥50% |
| Blood lymphocytes | <4000/μL | Decrease ≥50% | Increase ≥50% over |
| | | from baseline | baseline |
| Marrow‡ | Normocellular, <30% | 50% reduction in | |
| | lymphocytes, no B- | marrow infiltrate, or | |
| | lymphoid nodules. | B-lymphoid nodules | |
| | Hypocellular marrow | | |
| | defines CRi (5.1.6). | | |
| Group B | | | |
| Platelet count | >100 000/μL | >100 000/μL or | Decrease of ≥50% |
| | | increase ≥50% over | from baseline |
| | | baseline | secondary to CLL |
| Hemoglobin | >11.0 g/dL | >11 g/dL or | Decrease of >2 |
| | | increase ≥50% over | g/dL from baseline |
| | | baseline | secondary to CLL |
| Neutrophils^{‡} | >1500/μL | >1500/μL or >50% | |
| | | improvement over | |
| | | baseline | |

Group A criteria define the tumor load; Group B criteria define the function of the hematopoietic system (or marrow). CR (complete remission): all of the criteria have to be met, and patients have to lack disease-related constitutional symptoms; PR (partial remission): at least two of the criteria of group A plus one of the criteria of group B have to be met; SD is absence of progressive disease (PD) and failure to achieve at least a PR; PD: at least one of the above criteria of group A or group B has to be met. Sum of the products of multiple lymph nodes (as evaluated by CT scans in clinical trials, or by physical examination in general practice). These parameters are irrelevant for some response categories.

In certain embodiments, the treatment of a cancer may be assessed by Response Evaluation Criteria in Solid Tumors (RECIST 1.1) (see Thereasse P., et al. New Guidelines to Evaluate the Response to Treatment in Solid Tumors. J. of the National Cancer Institute; 2000; (92) 205-216 and Eisenhauer E. A., Therasse P., Bogaerts J., et al. New response evaluation criteria in solid tumours: Revised RECIST guideline (version 1.1). European J. Cancer; 2009; (45) 228-247). Overall responses for all possible combinations of tumor responses in target and non-target lesions with or without the appearance of new lesions are as follows:

| | Non-target | | Overall |
|---|---|---|---|
| Target lesions | lesions | New lesions | response |
| CR | CR | No | CR |
| CR | Incomplete | No | PR |
| | response/SD | | |
| PR | Non-PD | No | PR |
| SD | Non-PD | No | SD |
| PD | Any | Yes or no | PD |
| Any | PD | Yes or no | PD |
| Any | Any | Yes | PD |

CR = complete response; PR = partial response; SD = stable disease; and PD = progressive disease.

With respect to the evaluation of target lesions, complete response (CR) is the disappearance of all target lesions, partial response (PR) is at least a 30% decrease in the sum of the longest diameter of target lesions, taking as reference the baseline sum longest diameter, progressive disease (PD) is at least a 20% increase in the sum of the longest diameter of target lesions, taking as reference the smallest sum longest diameter recorded since the treatment started or the appearance of one or more new lesions and stable disease (SD) is neither sufficient shrinkage to qualify for partial response nor sufficient increase to qualify for progressive disease, taking as reference the smallest sum longest diameter since the treatment started.

With respect to the evaluation of non-target lesions, complete response (CR) is the disappearance of all non-target lesions and normalization of tumor marker level; incomplete response/stable disease (SD) is the persistence of one or more non-target lesion(s) and/or the maintenance of tumor marker level above the normal limits, and progressive disease (PD) is the appearance of one or more new lesions and/or unequivocal progression of existing non-target lesions.

The procedures, conventions, and definitions described below provide guidance for implementing the recommendations from the Response Assessment for Neuro-Oncology (RANO) Working Group regarding response criteria for high-grade gliomas (Wen P., Macdonald, D R., Reardon, D A., et al. Updated response assessment criteria for highgrade gliomas: Response assessment in neuro-oncology working group. J Clin Oncol 2010; 28:1963-1972). Primary modifications to the RANO criteria for Criteria for Time Point Responses (TPR) can include the addition of operational conventions for defining changes in glucocorticoid dose, and the removal of subjects' clinical deterioration component to focus on objective radiologic assessments. The baseline MRI scan is defined as the assessment performed at the end of the post-surgery rest period, prior to re-initiating compound treatment. The baseline MRI is used as the reference for assessing complete response (CR) and partial response (PR). Whereas, the smallest SPD (sum of the products of perpendicular diameters) obtained either at baseline or at subsequent assessments will be designated the nadir assessment and utilized as the reference for determining progression. For the 5 days preceding any protocol-defined MRI scan, subjects receive either no glucocorticoids or are on a stable dose of glucocorticoids. A stable dose is defined as the same daily dose for the 5 consecutive days preceding the MRI scan. If the prescribed glucocorticoid dose is changed in the 5 days before the baseline scan, a new baseline scan is required with glucocorticoid use meeting the criteria described above. The following definitions will be used.

Measurable Lesions: Measurable lesions are contrast-enhancing lesions that can be measured bidimensionally. A measurement is made of the maximal enhancing tumor diameter (also known as the longest diameter, LD). The greatest perpendicular diameter is measured on the same image. The cross hairs of bidimensional measurements should cross and the product of these diameters will be calculated.

Minimal Diameter: T1-weighted image in which the sections are 5 mm with 1 mm skip. The minimal LD of a measurable lesion is set as 5 mm by 5 mm. Larger diameters may be required for inclusion and/or designation as target lesions. After baseline, target lesions that become smaller than the minimum requirement for measurement or become no longer amenable to bidimensional measurement will be recorded at the default value of 5 mm for each diameter below 5 mm. Lesions that disappear will be recorded as 0 mm by 0 mm.

Multicentric Lesions: Lesions that are considered multicentric (as opposed to continuous) are lesions where there is normal intervening brain tissue between the two (or more) lesions. For multicentric lesions that are discrete foci of enhancement, the approach is to separately measure each enhancing lesion that meets the inclusion criteria. If there is no normal brain tissue between two (or more) lesions, they will be considered the same lesion.

Nonmeasurable Lesions: All lesions that do not meet the criteria for measurable disease as defined above will be considered non-measurable lesions, as well as all nonenhancing and other truly nonmeasurable lesions. Nonmeasurable lesions include foci of enhancement that are less than the specified smallest diameter (ie., less than 5 mm by 5 mm), nonenhancing lesions (eg., as seen on T1-weighted post-contrast, T2-weighted, or fluid-attenuated inversion recovery (FLAIR) images), hemorrhagic or predominantly cystic or necrotic lesions, and leptomeningeal tumor. Hemorrhagic lesions often have intrinsic T1-weighted hyperintensity that could be misinterpreted as enhancing tumor, and for this reason, the pre-contrast T1-weighted image may be examined to exclude baseline or interval sub-acute hemorrhage.

At baseline, lesions will be classified as follows: Target lesions: Up to 5 measurable lesions can be selected as target lesions with each measuring at least 10 mm by 5 mm, representative of the subject's disease; Non-target lesions: All other lesions, including all nonmeasurable lesions (including mass effects and T2/FLAIR findings) and any measurable lesion not selected as a target lesion. At baseline, target lesions are to be measured as described in the definition for measurable lesions and the SPD of all target lesions is to be determined. The presence of all other lesions is to be documented. At all post-treatment evaluations, the baseline classification of lesions as target and non-target lesions will be maintained and lesions will be documented and described in a consistent fashion over time (eg., recorded in the same order on source documents and eCRFs). All measurable and nonmeasurable lesions must be assessed using the same technique as at baseline (e.g., subjects should be imaged on the same MRI scanner or at least with the same magnet strength) for the duration of the study to reduce difficulties in interpreting changes. At each evaluation, target lesions will be measured and the SPD calculated. Non-target lesions will be assessed qualitatively and new lesions, if any, will be documented separately. At each evaluation, a time point response will be determined for target lesions, non-target lesions, and new lesion. Tumor progression can be established even if only a subset of lesions is assessed. However, unless progression is observed, objective status (stable disease, PR or CR) can only be determined when all lesions are assessed.

Confirmation assessments for overall time point responses of CR and PR will be performed at the next scheduled assessment, but confirmation may not occur if scans have an interval of <28 days. Best response, incorporating confirmation requirements, will be derived from the series of time points.

In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

Unless otherwise specified, to the extent that there is a discrepancy between a depicted chemical structure of a compound provided herein and a chemical name of a compound provided herein, the chemical structure shall control.

### Compound 1

The solid forms, formulations and methods of use provided herein relate to Compound 1:

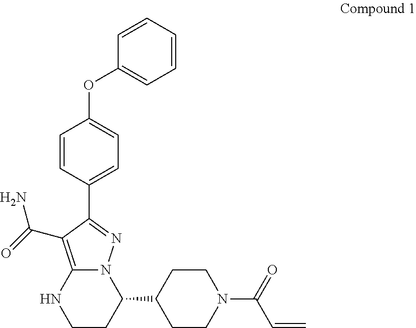 [SMILES: C=CC(=O)N1CCC([C@@H]2CCNc3c(C(N)=O)c(-c4ccc(Oc5ccccc5)cc4)nn32)CC1]

having the name(S)-7-(1-acryloylpiperidin-4-yl)-2-(4-phenoxyphenyl)-4,5,6,7-tetrahydropyrazolo[1,5-a]pyrimidine-3-carboxamide, including tautomers thereof.

Compound 1 is disclosed in U.S. Pat. No. 9,447,106, issued on Sep. 20, 2016, and International Pub. No. WO2014/173289, the entireties of each of which are incorporated by reference herein.

Crystalline Form A of Compound 1 is disclosed in U.S. Pat. No. 10,927,117, issued on Feb. 23, 2021, and International Pub. No. WO2018/033853, the entireties of each of which are incorporated by reference herein.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### Solid Form of Compound 1

While not intending to be bound by any particular theory, certain solid forms (e.g., salts, or cocrystals) are characterized by physical properties, e.g., stability, solubility, and dissolution rate, appropriate for pharmaceutical and therapeutic dosage forms. Moreover, while not wishing to be bound by any particular theory, certain solid forms (e.g., salts, or cocrystals) are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms (e.g., salts, or cocrystals) suitable for the manufacture of a solid dosage form. Such properties can be determined using particular analytical chemical techniques, including solid-state analytical techniques (e.g., X-ray diffraction, microscopy, spectroscopy, and thermal analysis), as described herein and known in the art.

In one embodiment, provided herein are solid forms (e.g., crystal forms, amorphous forms, salt form, or mixtures thereof) comprising (a) Compound 1; and (b) oxalic acid. In one embodiment, provided herein are solid forms (e.g., crystal forms, amorphous forms, or mixtures thereof) comprising (a) a free base of Compound 1; and (b) oxalic acid. Compound 1 can be synthesized or obtained according to a method known in the literature or based upon the teachings herein, including the methods described in detail in the examples herein. In certain embodiments, the coformer is oxalic acid.

In one embodiment, solid forms provided herein may be a crystal form, a salt form, or an amorphous form or mixtures thereof (e.g., mixtures of crystal forms, or mixtures of crystal and amorphous forms), which comprises (a) Compound 1; and (b) oxalic acid. In one embodiment, provided herein is a crystal form comprising (a) Compound 1; and (b) oxalic acid. In one embodiment, provided herein is a cocrystal comprising (a) Compound 1; and (b) oxalic acid. In one embodiment, provided herein is a salt form comprising (a) Compound 1; and (b) oxalic acid. In one embodiment, provided herein is an amorphous form comprising (a) Compound 1; and (b) oxalic acid. In one embodiment, provided herein is a mixture comprising (i) a cocrystal comprising (a) Compound 1; and (b) oxalic acid; and (ii) a crystal form of Compound 1. In one embodiment, provided herein is a mixture comprising (i) a salt comprising (a) Compound 1; and (b) oxalic acid; and (ii) a crystal form of Compound 1. In one embodiment, provided herein is a mixture comprising (i) a cocrystal comprising (a) Compound 1; and (b) oxalic acid; and (ii) an amorphous form of Compound 1. In one embodiment, provided herein is a mixture comprising (i) a salt comprising (a) Compound 1; and (b) oxalic acid; and (ii) an amorphous form of Compound 1.

In one embodiment, provided herein is a solid form comprising (a) Compound 1 and (b) oxalic acid that is substantially crystalline. In one embodiment, provided herein is a salt form comprising (a) Compound 1; and (b) oxalic acid that is substantially crystalline. In one embodiment, provided herein is a solid form comprising a cocrystal comprising (a) Compound 1 and (b) oxalic acid. In one embodiment, provided herein is a solid form comprising a salt comprising (a) Compound 1 and (b) oxalic acid. In one embodiment, provided herein is a solid form comprising (i) a cocrystal comprising (a) Compound 1 and (b) oxalic acid and (ii) an amorphous form of Compound 1. In one embodiment, provided herein is a solid form comprising (i) a salt comprising (a) Compound 1 and (b) oxalic acid and (ii) an amorphous form of Compound 1. In one embodiment, provided herein is a solid form comprising (i) a cocrystal comprising (a) Compound 1 and (b) oxalic acid and (ii) one or more additional crystal forms of Compound 1. In one embodiment, provided herein is a solid form comprising (i) a salt comprising (a) Compound 1 and (b) oxalic acid and (ii) one or more additional crystal forms of Compound 1.

In one embodiment, provided herein is an unsolvated solid form comprising (a) Compound 1 and (b) oxalic acid. In one embodiment, provided herein is an anhydrous solid form comprising (a) Compound 1 and (b) oxalic acid. In one embodiment, provided herein is an unsolvated crystal form comprising (a) Compound 1 and (b) oxalic acid. In one embodiment, provided herein is an anhydrous crystal form comprising (a) Compound 1 and (b) oxalic acid. In one embodiment, provided herein is an unsolvated amorphous form comprising (a) Compound 1 and (b) oxalic acid. In one embodiment, provided herein is an anhydrous amorphous form comprising (a) Compound 1 and (b) oxalic acid. In one embodiment, provided herein is a solvated solid form comprising (a) Compound 1 and (b) oxalic acid. In one embodiment, provided herein is a hydrated solid form comprising (a) Compound 1 and (b) oxalic acid (e.g., a hydrate having a stoichiometric or non-stoichiometric amount of water). In one embodiment, provided herein is a hydrated form of (a) Compound 1 and (b) oxalic acid, including, but not limited to, a hemihydrate, a monohydrate, a dihydrate, a trihydrate, and the like. In one embodiment, the hydrated form is substantially crystalline. In one embodiment, the hydrated form is substantially amorphous. In one embodiment, the anhydrous form is substantially crystalline. In one embodiment, the anhydrous form is substantially amorphous. In one embodiment, provided herein is an unsolvated cocrystal comprising (a) Compound 1 and (b) oxalic acid. In one embodiment, provided herein is an anhydrous cocrystal comprising (a) Compound 1 and (b) oxalic acid. In one embodiment, provided herein is a hydrated cocrystal comprising (a) Compound 1 and (b) oxalic acid. In one embodiment, provided herein is a solvated cocrystal comprising (a) Compound 1 and (b) oxalic acid.

In one embodiment, provided herein is an unsolvated salt comprising (a) Compound 1 and (b) oxalic acid. In one embodiment, provided herein is an anhydrous salt comprising (a) Compound 1 and (b) oxalic acid. In one embodiment, provided herein is a hydrated salt comprising (a) Compound 1 and (b) oxalic acid. In one embodiment, provided herein is a solvated salt comprising (a) Compound 1 and (b) oxalic acid. In one embodiment, the salt is substantially crystalline.

Solid forms provided herein can be prepared by the methods described herein, or by techniques, including, but not limited to, heating, cooling, freeze drying, spray drying, lyophilization, quench cooling the melt, rapid solvent evaporation, slow solvent evaporation, solvent recrystallization, antisolvent addition, slurry recrystallization, crystallization from the melt, desolvation, recrystallization in confined spaces, such as, e.g., in nanopores or capillaries, recrystallization on surfaces or templates, such as, e.g., on polymers, recrystallization in the presence of additives, such as, e.g., salt counter-molecules, cocrystal counter-molecules, desolvation, dehydration, rapid cooling, slow cooling, exposure to solvent and/or water, drying, including, e.g., vacuum drying, vapor diffusion, sublimation, grinding (including, e.g., cryo-grinding and solvent-drop grinding), microwave-induced precipitation, sonication-induced precipitation, laser-induced precipitation, and precipitation from a supercritical fluid. The particle size of the resulting solid forms, which can vary (e.g., from nanometer dimensions to millimeter dimensions), can be controlled, e.g., by varying crystallization conditions, such as, e.g., the rate of crystallization and/or the crystallization solvent system, or by particle-size reduction techniques, e.g., grinding, milling, micronizing, or sonication.

In some embodiments, the solid form comprising (a) Compound 1 and (b) oxalic acid can be obtained by crystallization from certain solvent systems, for example, solvent systems comprising one or more of the following solvents: ACN; EtOH; IPA; 1-BuOH; Butyl acetate; 1-Propanol; THF/H₂O, 1:1; MeOAc; MeOH/MTBE, 1:2; 1,4-Dioxane/H₂O, 2:1; CHCl₃/n-heptane, 1:1; DCM/toluene, 1:2; EtOAc; Anisole; CPME; DMSO/H₂O, 1:4; ACN/H₂O, 989:11, a_{w}˜0.2; ACN/H₂O, 978:22, a_{w}˜0.4; ACN/H₂O, 959:41, a_{w}˜0.6; ACN/H₂O, 925:75, a_{w}˜0.8; EtOAc saturated by water; isopropyl acetate; 2-methyltetrahydrofuran; or a combination thereof. In certain embodiments, a solid form provided herein (e.g., a cocrystal comprising (a) Compound 1 and (b) oxalic acid) can be obtained by cooling evaporation crystallization, powder in saturated solutions crystallization, slurry crystallization, and grinding crystallization.

In some embodiments, the cocrystal comprising (a) Compound 1 and (b) oxalic acid can be obtained by crystallization from certain solvent systems, for example, solvent systems comprising one or more of the following solvents: ACN; EtOH; IPA; 1-BuOH; Butyl acetate; 1-Propanol; THF/H₂O, 1:1; MeOAc; MeOH/MTBE, 1:2; CHC13/n-heptane, 1:1; DCM/toluene, 1:2; EtOAc; Anisole; CPME; DMSO/H₂O, 1:4; ACN/H₂O, 989:11, a_{w}˜0.2; ACN/H₂O, 978:22, a_{w}˜0.4; ACN/H₂O, 959:41, a_{w}˜0.6; ACN/H₂O, 925:75, a_{w}˜0.8; EtOAc saturated by water; isopropyl acetate; 2-methyltetrahydrofuran; or a combination thereof. In certain embodiments, a solid form provided herein (e.g., a cocrystal comprising (a) Compound 1 and (b) oxalic acid) can be obtained by cooling evaporation crystallization, powder in saturated solutions crystallization, slurry crystallization, and grinding crystallization.

In certain embodiments, the non-covalent forces are one or more hydrogen bonds (H-bonds). The coformer may be H-bonded directly to Compound 1 or may be H-bonded to an additional molecule which is bound to Compound 1. The additional molecule may be H-bonded to Compound 1 or bound ionically or covalently to Compound 1. The additional molecule could also be a different active or inactive ingredient. In certain embodiments, the cocrystals may include one or more solvate molecules in the crystalline lattice, i.e., solvates of cocrystals, or a cocrystal further comprising a solvent or compound that is a liquid at room temperature. In certain embodiments, the cocrystals may be a cocrystal between a coformer and a salt of Compound 1. In certain embodiments, the non-covalent forces are pi-stacking, guest-host complexation and/or van der Waals interactions. Hydrogen bonding can result in several different intermolecular configurations. For example, hydrogen bonds can result in the formation of dimers, linear chains, or cyclic structures. These configurations can further include extended (two-dimensional) hydrogen bond networks and isolated triads. In certain embodiments, the coformer is a solid under ambient temperature conditions when in its pure form. In one embodiment, the coformer is oxalic acid.

In certain embodiments, cocrystals formation can lead to enhancement of physical properties of the resulting solid forms, such as solubility, dissolution rate, bioavailability, physical stability, chemical stability, flowability, fractability, or compressibility. In certain embodiments, salts formation can lead to enhancement of physical properties of the resulting solid forms, such as solubility, dissolution rate, bioavailability, physical stability, chemical stability, flowability, fractability, or compressibility.

In certain embodiments, provided herein are cooling evaporative methods for making a solid form of Compound 1, comprising 1) obtaining a close-to saturated solution of Compound 1 and oxalic acid in a ratio (e.g., about 1:0.2 to about 1:3, about 1:0.5, about 1:0.55, about 1:0.9, about 1:1, about 1:1.2, and about 1:2.2) in a solvent; 2) heating the solution to a first temperature (e.g., about 30° C. to about 50° C.); 3) cooling the solution to a second temperature (e.g., about −5° C. to about 15° C.); 4) keeping the solution at the second temperature for a period of time (e.g., 72 hours); 5) filtering the solution to yield a solid if there is precipitation; and 6) evaporating the solvent to collect a solid if there is no precipitation after step 4. In one embodiment, the solution may be seeded. In certain embodiments, provided herein are cooling evaporative methods for making a solid form of Compound 1, comprising 1) obtaining a close-to saturated solution of Compound 1 and oxalic acid in a solvent; 2) heating the solution to about 40° C.); 3) cooling the solution to about 2° C.); 4) keeping the solution at about 2° C. for about 48 hours; 5) filtering the solution to yield a solid if there is precipitation; and 6) evaporating the solvent to collect a solid if there is no precipitation after step 4. In certain embodiments, the solvent is ACN; EtOH; IPA; 1-BuOH; Butyl acetate; 1-Propanol; THF/H₂O, 1:1; MeOAc; MeOH/MTBE, 1:2; 1,4-Dioxane/H₂O, 2:1; CHC13/n-heptane, 1:1; DCM/toluene, 1:2; EtOAc; Anisole; CPME; DMSO/H₂O, 1:4; ACN/H₂O, 989:11, a_{w}˜0.2; ACN/H₂O, 978:22, a_{w}˜0.4; ACN/H₂O, 959:41, a_{w}˜0.6; ACN/H₂O, 925:75, a_{w}˜0.8; EtOAc saturated by water; isopropyl acetate; 2-methyltetrahydrofuran; or a combination thereof. In one embodiment, the molar ratio of Compound 1 and the oxalic acid in step 1 is about 1:0.5. In one embodiment, the solid form is a cocrystal. In one embodiment, the solid form is a salt.

In certain embodiments, provided herein are powder in saturated solutions crystallization methods for making a solid form of Compound 1, comprising 1) obtaining a saturated solution of Compound 1 in a solvent; 2) adding oxalic acid into the solution; 3) stirring the solution at ambient temperature for a period of time; 4) filtering the solution to yield a first solid and 5) evaporate the solvent to collect a second solid. In one embodiment, the solution may be seeded. In certain embodiments, the solvent is ACN; EtOH; IPA; 1-BuOH; Butyl acetate; 1-Propanol; THF/H₂O, 1:1; MeOAc; MeOH/MTBE, 1:2; 1,4-Dioxane/H₂O, 2:1; CHCl₃/n-heptane, 1:1; DCM/toluene, 1:2; EtOAc; Anisole; CPME; DMSO/H₂O, 1:4; ACN/H₂O, 989:11, a_{w}˜0.2; ACN/H₂O, 978:22, a_{w}˜0.4; ACN/H₂O, 959:41, a_{w}˜0.6; ACN/H₂O, 925:75, a_{w}˜0.8; EtOAc saturated by water; isopropyl acetate; 2-methyltetrahydrofuran; or a combination thereof. In one embodiment, the molar ratio of Compound 1 and oxalic acid is about 1:0.5. In one embodiment, the period of time is about 72 hours. In one embodiment, the solid form is a cocrystal. In one embodiment, the solid form is a salt.

In certain embodiments, provided herein are slurry methods for making a solid form of Compound 1, comprising 1) obtaining a slurry of Compound 1 and oxalic acid in a ratio in a solvent; 2) stirring the slurry for a period of time; 3) collecting a solid from the slurry by filtration (e.g., centrifuge filtration). In one embodiment, the solution may be seeded. In certain embodiments, the solvent is ACN; EtOH; IPA; 1-BuOH; Butyl acetate; 1-Propanol; THF/H₂O, 1:1; MeOAc; MeOH/MTBE, 1:2; 1,4-Dioxane/H₂O, 2:1; CHCl₃/n-heptane, 1:1; DCM/toluene, 1:2; EtOAc; Anisole; CPME; DMSO/H₂O, 1:4; ACN/H₂O, 989:11, a_{w}˜0.2; ACN/H₂O, 978:22, a_{w}˜0.4; ACN/H₂O, 959:41, a_{w}˜0.6; ACN/H₂O, 925:75, a_{w}˜0.8; EtOAc saturated by water; isopropyl acetate; 2-methyltetrahydrofuran; or a combination thereof. In one embodiment, the molar ratio of Compound 1 and oxalic acid is about 1:0.5. In one embodiment, the period of time is about 3 days. In one embodiment, the solid form is a cocrystal. In one embodiment, the solid form is a salt.

In certain embodiments, provided herein are grinding methods for making a solid form of Compound 1, comprising 1) adding Compound 1, oxalic acid, and a solvent into a grinding machine; 2) shaking the container for a period of time at a particular frequency; 3) collecting the resulting solid by filtration (e.g., centrifuge filtration). In certain embodiments, the solvent is ACN; EtOH; IPA; 1-BuOH; Butyl acetate; 1-Propanol; THF/H₂O, 1:1; MeOAc; MeOH/MTBE, 1:2; 1,4-Dioxane/H₂O, 2:1; CHC13/n-heptane, 1:1; DCM/toluene, 1:2; EtOAc; Anisole; CPME; DMSO/H₂O, 1:4; ACN/H₂O, 989:11, a_{w}˜0.2; ACN/H₂O, 978:22, a_{w}˜0.4; ACN/H₂O, 959:41, a_{w}˜0.6; ACN/H₂O, 925:75, a_{w}˜0.8; EtOAc saturated by water; isopropyl acetate; 2-methyltetrahydrofuran; or a combination thereof. In one embodiment, the molar ratio of Compound 1 and oxalic acid is about 1:0.5. In one embodiment, the period of time is about 72 hour. In one embodiment, the frequency is about 30 Hz. In one embodiment, the solid form is a cocrystal. In one embodiment, the solid form is a salt.

The solid form cocrystals provided herein (e.g., Form 1) may be characterized using a number of methods known to a person having ordinary skill in the art, including, but not limited to, single crystal X-ray diffraction, X-ray powder diffraction (XRPD), microscopy (e.g., scanning electron microscopy (SEM)), thermal analysis (e.g., differential scanning calorimetry (DSC), thermal gravimetric analysis (TGA), and hot-stage microscopy), spectroscopy (e.g., infrared, Raman, and solid-state nuclear magnetic resonance), single differential thermal analysis (SDTA), high performance liquid chromatography coupled with mass spectroscopy (HPLC-MS), thermogravimetrical analysis coupled with single differential thermal analysis (TGA-SDTA), and thermogravimetric analysis coupled with mass spectroscopy (TGA-MS). The particle size and size distribution of the solid form provided herein may be determined by conventional methods, such as laser light scattering technique.

The purity of the solid form provided herein may be determined by standard analytical methods, such as thin layer chromatography (TLC), gel electrophoresis, gas chromatography, high performance liquid chromatography (HPLC), and mass spectrometry (MS).

It should be understood that the numerical values of the peaks of an X-ray powder diffraction pattern may vary slightly from one machine to another or from one sample to another, and so the values quoted are not to be construed as absolute, but with an allowable variability, such as ±0.2 degrees two theta (° 2θ) (see United State Pharmacopoeia, page 2228 (2003)).

### Solid Form 1 Comprising Compound 1 and Oxalic Acid

Provided herein is solid Form 1, comprising Compound 1 and oxalic acid. In one embodiment, Form 1 is a cocrystal. In one embodiment, Form 1 is a salt. In one embodiment, Form 1 is substantially crystalline. In one embodiment, provided herein is a solid form comprising (i) a cocrystal comprising Compound 1 and oxalic acid and (ii) an amorphous form of Compound 1. In one embodiment, provided herein is a solid form comprising (i) a salt comprising Compound 1 and oxalic acid and (ii) an amorphous form of Compound 1. In one embodiment, provided herein is a solid form comprising (i) a cocrystal comprising Compound 1 and oxalic acid and (ii) one or more additional crystal forms of Compound 1. In one embodiment, provided herein is a solid form comprising (i) a salt comprising Compound 1 and oxalic acid and (ii) one or more additional crystal forms of Compound 1. Provided herein are various embodiments, preparations, or modifications of Form 1 comprising Compound 1 and oxalic acid.

In one embodiment, Form 1 is an anhydrous form comprising Compound 1 and oxalic acid. In one embodiment, Form 1 is a solvated form comprising Compound 1 and oxalic acid. In one embodiment, Form 1 is an ACN solvated form comprising Compound 1 and oxalic acid. In another embodiment, Form 1 is crystalline.

In one embodiment, the molar ratio of oxalic acid to Compound 1 of Form 1 is 0.5±0.1, 0.5±0.05, 0.5±0.01, or about 0.5. In one embodiment, Form 1 is an oxalic acid co-crystal form. In one embodiment, Form 1 is an oxalic acid salt of Compound 1.

In certain embodiments, provided herein are slurry methods for making Form 1, comprising 1) obtaining a slurry of Compound 1 and oxalic acid in a ratio in a solvent; 2) stirring the slurry for a period of time; 3) collecting a solid from the slurry by filtration (e.g., centrifuge filtration). In one embodiment, the solution may be seeded. In certain embodiments, the solvent is ACN; EtOH; IPA; 1-BuOH; Butyl acetate; 1-Propanol; THF/H₂O, 1:1; MeOAc; MeOH/MTBE, 1:2; 1,4-Dioxane/H₂O, 2:1; CHC13/n-heptane, 1:1; DCM/toluene, 1:2; EtOAc; Anisole; CPME; DMSO/H₂O, 1:4; ACN/H₂O, 989:11, a_{w}˜0.2; ACN/H₂O, 978:22, a_{w}˜0.4; ACN/H₂O, 959:41, a_{w}˜0.6; ACN/H₂O, 925:75, a_{w}˜0.8; EtOAc saturated by water; isopropyl acetate; 2-methyltetrahydrofuran; or a combination thereof. In one embodiment, the molar ratio of Compound 1 and oxalic acid is about 1:0.3 to 1:3. In one embodiment, the molar ratio of Compound 1 and oxalic acid is about 1:0.5, about 1:1.2, about 1:0.55, about 1:2.2, or about 1:1. In one embodiment, the period of time is about 3 days. In one embodiment, the solid form is a cocrystal. In one embodiment, the solid form is a salt.

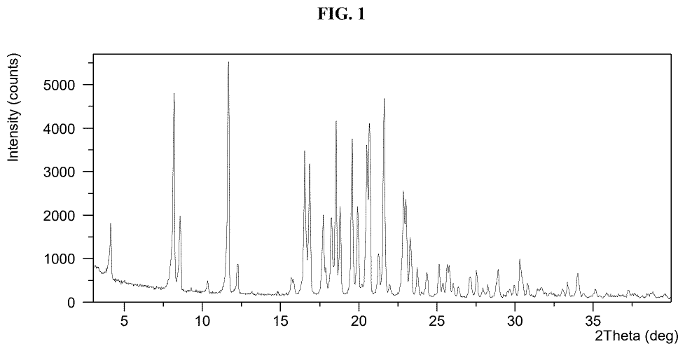

In certain embodiments, a solid form provided herein, e.g., Form 1, is substantially crystalline, as indicated by, e.g., X-ray powder diffraction measurements. In one embodiment, Form 1 of Compound 1 has an X-ray powder diffraction pattern substantially as shown in FIG. 1. In one embodiment, Form 1 of Compound 1 has one or more characteristic X-ray powder diffraction peaks at a two-theta angle of approximately 8.17, 11.65, or 21.61 degrees as depicted in FIG. 1. In another embodiment, Form 1 of Compound 1 has one, two, three, four, or five characteristic X-ray powder diffraction peaks at a two-theta angle of approximately 8.17, 11.65, 18.54, 19.56, or 21.61, degrees. In another embodiment, Form 1 of Compound 1 has one, two, three, four, five, or six characteristic X-ray powder diffraction peaks at a two-theta angle of approximately 8.17, 8.56, 11.65, 18.54, 19.56, or 21.61, degrees. In another embodiment, Form 1 of Compound 1 has one, two, three, four, five, six, seven, eight, or nine characteristic X-ray powder diffraction peaks at a two-theta angle of approximately 8.17, 8.56, 11.65, 16.52, 16.84, 18.54, 19.56, 19.92, or 21.61, degrees. In another embodiment, Form 1 of Compound 1 has one, two, three, four, five, six, seven, or eight characteristic X-ray powder diffraction peaks as set forth in Table 9.

In one embodiment, Form 1 of Compound 1 has one or more characteristic X-ray powder diffraction peaks at a two-theta angle of approximately 8.17, 11.65, or 21.61 degrees as depicted in FIG. 1. In another embodiment, Form 1 of Compound 1 has one, two, three, four, five, or six characteristic X-ray powder diffraction peaks at a two-theta angle of approximately 8.17, 11.65, 18.54, 19.56, 20.69, or 21.61 degrees.

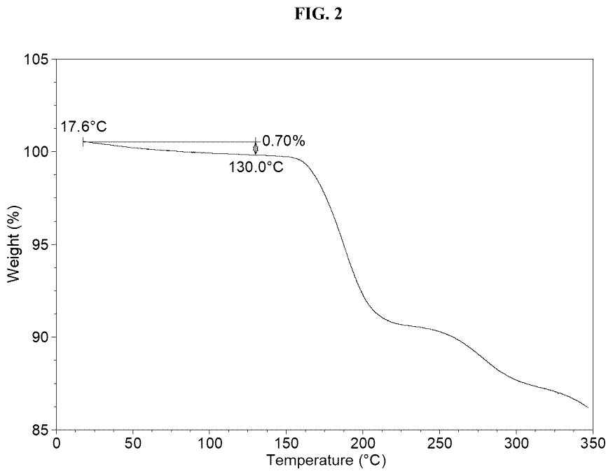

In one embodiment, provided herein is Form 1 having a thermogravimetric (TGA) thermograph corresponding substantially to the representative TGA thermogram as depicted in FIG. 2. In certain embodiments, the crystalline form exhibits a TGA thermogram comprising a total mass loss of approximately 0.7% of the total mass of the sample between approximately 17.6° C. and approximately 130.0° C. when heated from approximately 17.6° C. to approximately 300° C. Thus, in certain embodiments, the crystalline form loses about 0.7% of the total mass of the crystal form when heated from about 17.6° C. to about 130° C.

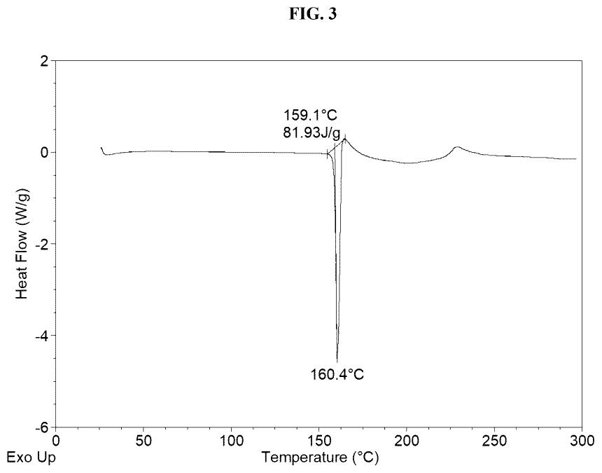

In one embodiment, provided herein is Form 1 having a differential scanning calorimetry (DSC) thermogram as depicted in FIG. 3 comprising an endothermic event with a maximum at about 160.4° C., when heated from approximately 25° C. to approximately 300° C. In one embodiment, the crystal form has a differential scanning calorimetry thermogram comprising an endothermic event with an onset temperature at approximately 159.1° C., when heated from about 50° C. to about 200° C.

In still another embodiment, Form 1 of Compound 1 is substantially pure. In certain embodiments, the substantially pure Form 1 of Compound 1 is substantially free of other solid forms, e.g., amorphous form. In certain embodiments, the purity of the substantially pure Form 1 of Compound 1 is no less than about 95% pure, no less than about 96% pure, no less than about 97% pure, no less than about 98% pure, no less than about 98.5% pure, no less than about 99% pure, no less than about 99.5% pure, or no less than about 99.8% pure.

### Methods of Use

The solid forms and the pharmaceutical compositions provided herein can be used in all the methods provided herein. The solid forms and the pharmaceutical compositions provided herein can be used in the treatment of all diseases, disorders or conditions provided herein.

Provided herein are methods for treating or preventing a cancer, comprising administering a solid form of Compound 1 provided herein or a pharmaceutical composition thereof to a patient having a cancer.

In some embodiments, the cancer is B-cell proliferative disease. For example, B-cell proliferative disease is chronic lymphocytic, non-Hodgkin's lymphoma, diffuse large B cell lymphoma, marginal zone lymphoma (MZL), mantle cell lymphoma (MCL), Waldenström's macroglobulinemia (WM), follicular lymphoma (FL), chronic lymphocytic leukemia (CLL), or a combination of two or more thereof. In some such embodiments, the cancer has progressed on standard anti-cancer therapy, or the patient is not able to tolerate standard anti-cancer therapy. In yet others, the cancer is a cancer for which no approved therapy exists. In some embodiments, the cancer is resistant to standard therapy. In another, the patient has relapsed after standard therapy.

In certain embodiments, the cancer is a chronic lymphocytic leukemia (CLL).

In certain embodiments, the cancer is a marginal zone lymphoma (MZL).

In certain embodiments, the cancer is a Waldenström's macroglobulinemia (WM).

In certain embodiments, the cancer is Mantle cell lymphoma (MCL).

In certain embodiments, the cancer is follicular lymphoma (FL).

The B-cell proliferative disease is chronic lymphocytic leukemia/small lymphocytic lymphoma (CLL/SLL).

In certain embodiments, the cancer is a cancer of the head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, and brain or central nervous system.

In other embodiments, the cancer is a solid tumor. In certain embodiments, the solid tumor is a relapsed or refractory solid tumor.

In other embodiments, the solid tumor can be an advanced solid tumor.

In other embodiments, the cancer is a cancer associated with the pathways involving BTK.

In certain embodiments, provided herein are methods for achieving a Response Evaluation Criteria in Solid Tumors (for example, RECIST 1.1) of complete response, partial response or stable disease in a patient having a solid tumor, comprising administering a solid form of Compound 1 provided herein or a pharmaceutical composition thereof to said patient. In certain embodiments, provided herein are methods for achieving a National Cancer Institute-Sponsored Working Group on Chronic Lymphocytic Leukemia (NCI-WG CLL) of complete response, partial response or stable disease in a patient having leukemia, comprising administering a solid form of Compound 1 provided herein or a pharmaceutical composition thereof to said patient. In certain embodiments, provided herein are methods for achieving a Prostate Cancer Working Group 2 (PCWG2) Criteria of complete response, partial response or stable disease in a patient having prostate cancer, comprising administering a solid form of Compound 1 provided herein or a pharmaceutical composition thereof to said patient. In certain embodiments, provided herein are methods for achieving an International Workshop Criteria (IWC) for non-Hodgkin's lymphoma of complete response, partial response or stable disease in a patient having non-Hodgkin's lymphoma, comprising administering a solid form of Compound 1 provided herein or a pharmaceutical composition thereof to said patient. In certain embodiments, provided herein are methods for achieving an International Uniform Response Criteria (IURC) for multiple myeloma of complete response, partial response or stable disease in a patient having multiple myeloma, comprising administering a solid form of Compound 1 provided herein or a pharmaceutical composition thereof to said patient.

In certain embodiments, provided herein are methods for increasing survival without disease progression of a patient having a cancer, comprising administering a solid form of Compound 1 provided herein or a pharmaceutical composition thereof to said patient.

In certain embodiments, provided herein are methods for treating a cancer, the methods comprising administering a solid form of Compound 1 provided herein or a pharmaceutical composition thereof to a patient having a cancer, wherein the treatment results in prevention or retarding of clinical progression, such as cancer-related cachexia or increased pain.

In some embodiments, provided herein are methods for treating a cancer, the methods comprising administering a solid form of Compound 1 provided herein or a pharmaceutical composition thereof to a patient having a cancer, wherein the treatment results in one or more of inhibition of disease progression, increased Time To Progression (TTP), increased Progression Free Survival (PFS), and/or increased Overall Survival (OS), among others.

### Pharmaceutical Compositions

Solid forms of Compound 1 provided herein are useful for the preparation of pharmaceutical compositions, comprising an effective amount of a solid form of Compound 1 and a pharmaceutically acceptable carrier or vehicle. In some embodiments, the pharmaceutical compositions described herein are suitable for oral, parenteral, mucosal, transdermal, or topical administration.

In certain embodiments, provided herein are compositions comprising one or more solid forms of Compound 1. Also provided herein are compositions comprising: (i) one or more solid forms of Compound 1 provided herein (e.g., one or more cocrystal forms or mixtures thereof), and (ii) other active or inactive ingredient(s).

In one embodiment, the pharmaceutical compositions provided herein comprise a solid form of Compound 1 and one or more pharmaceutically acceptable excipients or carriers. In one embodiment, the pharmaceutical compositions provided herein comprise Form 1 of Compound 1 and one or more pharmaceutically acceptable excipients or carriers.

In one embodiment, the pharmaceutically acceptable excipients and carriers are selected from binders, diluents, disintegrants and lubricants. In another embodiment, the pharmaceutically acceptable excipients and carriers further include one or more antioxidants (e.g., EDTA or BHT).

In certain embodiments, the binders include, but are not limited to, cellulose (e.g., microcrystalline cellulose, such as AVICEL® PH 101, AVICEL® PH112, and AVICEL® PH 102) and starch (e.g., pregelatinized starch (STARCH 1500®)). In one embodiment, the binder is cellulose. In another embodiment, the binder is microcrystalline cellulose. In yet another embodiment, the binder is AVICEL® PH 101. In yet another embodiment, the binder is AVICEL® PH 102. In yet another embodiment, the binder is starch. In yet another embodiment, the binder is pregelatinized starch. In still another embodiment, the binder is STARCH 1500®.

In certain embodiments, the diluents include, but are not limited to, lactose (e.g., lactose monohydrate (FAST FLO® 316) and lactose anhydrous), cellulose (e.g., microcrystalline cellulose, such as AVICEL® PH 101 and AVICEL® PH 102). In one embodiment, the diluent is lactose. In another embodiment, the diluent is lactose monohydrate. In yet another embodiment, the diluent is FAST FLO® 316. In yet another embodiment, the diluent is lactose anhydrous. In yet another embodiment, the diluent is cellulose. In yet another embodiment, the diluent is microcrystalline cellulose. In yet another embodiment, the diluent is AVICEL® PH 101. In still another embodiment, the diluent is AVICEL® PH 102).

In certain embodiments, the disintegrants include, but are not limited to, starch (e.g., corn starch) and carboxymethyl cellulose (e.g., croscarmellose sodium, such as AC-DI-SOL®). In one embodiment, the disintegrant is starch. In another embodiment, the disintegrant is corn starch. In yet another embodiment, the disintegrant is carboxymethyl cellulose. In yet another embodiment, the disintegrant is croscarmellose sodium. In still another embodiment, the disintegrant is AC-DI-SOL®.

In certain embodiments, the lubricants include, but are not limited to, starch (e.g., corn starch), magnesium stearate, and stearic acid. In one embodiment, the lubricant is starch. In another embodiment, the lubricant is corn starch. In yet another embodiment, the lubricant is magnesium stearate. In still another embodiment, the lubricant is stearic acid.

In another embodiment, the pharmaceutical compositions provided herein comprise a solid form of Compound 1 and one or more pharmaceutically acceptable excipients or carriers, each independently selected from carboxymethyl cellulose, cellulose, lactose, magnesium stearate, starch, and stearic acid.

In certain embodiments, provided herein are pharmaceutical compositions, wherein the amounts of the recited components can independently be varied by 1%, 2%, 3%, 4%, 5%, 6%, 7%, 8%, 9%, 10%, 15%, 20% or 25%.

The pharmaceutical compositions provided herein can be provided in a unit-dosage form or multiple-dosage form. A unit-dosage form, as used herein, refers to physically discrete unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an individually packaged tablet or capsule. A unit-dosage form may be administered in fractions or multiples thereof. A multiple-dosage form is a plurality of identical unit-dosage forms packaged in a single container to be administered in segregated unit-dosage form. Dosage amounts are expressed herein as free base equivalent amounts unless the context requires otherwise.

In another embodiment, provided herein are unit dosage formulations that comprise between about 0.1 mg and about 2000 mg, about 1 mg and 200 mg, about 35 mg and about 1400 mg, about 125 mg and about 1000 mg, about 250 mg and about 1000 mg, or about 500 mg and about 1000 mg solid form of Compound 1, or a pharmaceutically acceptable salt, isotopologue or solid form thereof.

In a particular embodiment, provided herein are unit dosage formulation comprising about 0.1 mg, about 0.25 mg, about 0.5 mg, about 1 mg, about 2 mg, about 2.5 mg, about 5 mg, about 7.5 mg, about 8 mg, about 10 mg, about 15 mg, about 20 mg, about 25 mg, about 30 mg, about 35 mg, about 40 mg, about 45 mg, about 50 mg, about 60 mg, about 70 mg, about 75 mg, about 100 mg, about 125 mg, about 140 mg, about 150 mg, about 175 mg, about 200 mg, about 250 mg, about 280 mg, about 300 mg, about 350 mg, about 400 mg, about 500 mg, about 560 mg, about 600 mg, about 700 mg, about 750 mg, about 800 mg, about 1000 mg or about 1400 mg of a solid form of Compound 1. In a particular embodiment, provided herein are unit dosage formulations that comprise about 2.5 mg, about 5 mg, about 7.5 mg, about 8 mg, about 10 mg, about 15 mg, about 20 mg, about 25 mg, about 30 mg, about 40 mg, about 45 mg, about 50 mg, about 60 mg or about 100 mg of a solid form of Compound 1, or a pharmaceutically acceptable salt, tautomer, isotopologue or stereoisomer thereof.

In some embodiments, a unit dosage form comprising Compound 1, or a pharmaceutically acceptable salt, isotopologue or solid form thereof can be administered once daily (QD), twice daily (BID), three times daily, four times daily or more often.

In certain embodiments, provided herein are methods for preparing a composition provided herein, comprising: (i) weighing out the desired amount of a solid form of Compound 1 (e.g., Form 1) and the desired amount of excipients (such as lactose monohydrate, croscarmellose sodium and/or microcrystalline cellulose); (ii) mixing or blending the solid form of Compound 1 and the excipients; (iii) passing the mixture of the solid form of Compound 1 and excipients through a screen (such as a 25 mesh screen); (iv) mixing or blending the solid form of Compound 1 and the excipients after passage through the screen; (v) weighing out the desired amount of lubricating agents (such as stearic acid and magnesium stearate); (vi) passing the lubricating agents through a screen (such as a 35 mesh screen); (vii) mixing or blending the solid form of Compound 1, the excipients and the lubricating agents; (viii) compressing the mixture of the solid form of Compound 1, the excipients and the lubricating agents (such as into a tablet form); and optionally (ix) coating the compressed mixture of the solid form of Compound 1 thereof, the excipients and the lubricating agents with a coating agent (such as Opadry pink, yellow or beige). In certain embodiments, the methods for preparing a composition provided herein are carried out in the dark, under yellow light or in the absence of UV light.

In certain embodiments, the pharmaceutical compositions provided herein comprise Form 1 of Compound 1, including substantially pure Form 1.

### EXAMPLES

The following Examples are presented by way of illustration, not limitation. The following abbreviations are used in descriptions and examples:
2MXETOH: 2-Methoxyethanol
AAC: Accelerated aging conditions (48 hours at 40° C. and 75% RH)
ACN: Acetonitrile
Am: Amorphous
AmPhos: p-Dimethylamino phenylditbutylphosphine
API: Active Pharmaceutical Ingredient
AS: ID for anti-solvent crystallization experiment
Boc: tert-Butoxycarbonyl
dba: Dibenzylidene acetone
DCM: Dichloromethane
DIPEA: N,N-Diisopropylethylamine
DMF: N,N-Dimethylformide
DMSO: Dimethylsulfoxide
DSC: Differential Scanning calorimetry
ECP: ID for evaporative experiment
EDTA: Ethylenediamine tetraacetate
ESI: Electronspray ionization
EtOH: Ethanol
FTIR: Fourier Transform Infra Red Spectroscopy
GRP: Grinding experiment
HF: ID for hot-filtration crystallization experiment
HPLC: High performance liquid chromatography
IPA: 2-Propanol
LCMS: Liquid Chromatography with Mass Spectroscopy
MeOH: Methanol
mp: Melting point
MS: Mass spectrometry
Ms: Mesylate or methanesulfonyl
MTBE: tert-Butyl methyl ether
MTBE: methyl tert-butyl ether
NBS: N-Bromosuccinimide
NMP: N-Methyl-2-pyrrolidone
NMP: N-methylpyrrolidinone
NMR: Nuclear magnetic resonance
PSU: ID for cooling-evaporative crystallization experiment
QSA: ID for Phase 1 experiments
RH: Relative Humidity
RT: Room Temperature
S: Solvent
SDTA: Single Differential Thermal Analysis
SLP: ID for slurry experiment
SM: Starting material
TA: Thermal Analysis
TCP: ID for thermocycling and reflux experiment
Tf: triflate or trifluoromethanesulfonyl
TFA: Trifluoroacetic acid
TFE: 2,2,2-Trifluoroethanol
TGA: Thermogravimetric Analysis
TGA-MS/TG-MS: Thermogravimetric Analysis coupled with Mass Spectroscopy
THF: Tetrahydrofuran
TLC: Thin layer chromatography
VDL: ID for vapor diffusion into solutions experiment
VDS: ID for vapor diffusion onto solids experiment
XRPD: X-Ray Powder Diffraction

### Analytical Methods

### XRPD

For XRPD analysis, a PANalytical Empyrean X-ray powder diffract meter was used. The XRPD parameters used are listed in Table 1.

TABLE 1
Parameters for XRPD test

| Parameters | XRPD |
|---|---|
| Model | Empyrean |
| Test mode | Reflection |
| X-Ray wavelength | Cu, kα, |
| | Kα1 (Å): 1.540598, |
| | Kα2 (Å): 1.544426 |
| | Kα2/Kα1 intensity ratio: 0.50 |
| X-Ray tube setting | 45 kV, 40 mA |
| Divergence slit | ⅛° |
| Scan mode | Continuous |
| Scan range (°2TH) | 3-40 |
| Scan step time (s) | 46.7 |
| Step size (°2TH) | 0.0263 |
| Test Time | ~5 min |

### TGA and DSC

TGA data was collected using a TA Q5000/Discovery TGA 5500 from TA Instruments. DSC was performed using a Discovery DSC 2500 from TA Instruments. TGA and DSC have a standard deviation of about ±0.3° C. Detailed parameters used are listed in Table 2.

TABLE 2
Parameters for TGA and DSC test

| Parameters | TGA | DSC |
|---|---|---|
| Method | Ramp | Ramp |
| Sample pan | Aluminum, open | Aluminum, crimped |
| Temperature | RT-350° C. | 25° C.-300° C. |
| Heating rate | 10° C./min | 10° C./min |
| Purge gas | N₂ | N₂ |

### HPLC

Waters H-Class was utilized, and detailed chromatographic condition is listed in Table 3.

TABLE 3
Chromatographic conditions and parameters
for purity and solubility test

| HPLC | Agilent 1260 with DAD detector |
|---|---|
| Column | Waters Xbridge C18, 150 × 4.6 mm, 5 μm |
| Mobile phase | A: 0.1% TFA in H₂O |
| | B: 0.1% TFA in acetonitrile |

| | |
|---|---|
| Purity | Solubility |

| Gradient table | Time (min) | % B | Time (min) | % B |
|---|---|---|---|---|
| | 0.0 | 10 | 0.0 | 10 |
| | 16.0 | 90 | 5.0 | 90 |
| | 18.0 | 90 | 6.0 | 90 |
| | 18.1 | 10 | 6.1 | 10 |
| | 23.0 | 10 | 10.0 | 10 |

| Run time | 23.0 | min | 10.0 | min |
|---|---|---|---|---|
| Post time | 0.0 | min | 0.0 | min |
| Flow rate | 1.0 | mL/min | 1.0 | mL/min |
| Injection volume | 5 | μL | 5 | μL |
| Detector | UV at 238 nm | | UV at 238 nm | |
| wavelength | | | | |
| Column | 40° | C. | 40° | C. |

| | | |
|---|---|---|
| temperature | | |
| Sampler | RT | RT |
| temperature | | |
| Diluent | ACN:H₂O (1:1, v/v) | ACN:H₂O (1:1, v/v) |

### IC

Thermo Scientific™ Dionex™ Aquion™ Ion Chromatography (IC) System 1100 with conductivity detector was utilized and detailed chromatographic condition is listed in Table 4.

TABLE 4
Chromatographic conditions and parameters for ion content test

| Parameters | ThermoFisher ICS-1100 | |
|---|---|---|
| Column | IonPac AS18 Analytical Column (4 × 250 mm) | |
| Mobile phase | 25 mM NaOH | |
| Injection volume | 25 | μL |
| Flow rate | 1.0 | mL/min |
| Cell Temp. | 35° | C. |
| Column Temp. | 35° | C. |
| Current | 80 | mA |
| Run Time | 16 | mins |

### Experimental Methods

Table 5 provides a summary of the solvents and methods for each batch.

TABLE 5
Summary of Solvents and Methods for Each Batch

| Batch No | Solvent | Method | Scale | |
|---|---|---|---|---|
| Trial 1 | Acetonitrile | Slurry at RT | 800 | mg |
| Trial 2 | Acetonitrile | slurry with seed at RT | 400 | mg |
| Trial 3 | Acetonitrile | Slurry at RT | 40 | mg |
| Trial 4 | Acetonitrile | Slurry at RT | 40 | mg |
| Trial 5 | Acetonitrile | Slurry at RT | 40 | mg |
| Trial 6 | Isopropyl Acetate | Slurry at RT | 40 | mg |
| Trial 7 | 2-Methyltetrahydrofuran | Slurry at RT | 40 | mg |
| Trial 8 | Acetonitrile | Temperature cycling | 15 | mg |
| Trial 9 | Isopropyl Acetate | Temperature cycling | 15 | mg |
| Trial 10 | 2-Methyltetrahydrofuran | Temperature cycling | 15 | mg |

### Preparation Procedures of Oxalate Type A

### Trial 1

Weigh 800.0 mg freebase Form A, suspended with 4 mL ACN into an HPLC vial.
Dissolve 106.9 mg oxalic acid dehydrate (0.5 eq.) with 2.5 mL Acetonitrile (ACN).
Add the acid solution into the freebase suspension. During the addition, the mixture formed a clear solution and the solids crashed out in seconds after acid addition.
Magnetically stir at RT for 1 day.
Solids were isolated from the suspension by centrifugation. The solids were vacuum-dried at RT overnight before characterization.

### Trial 2

Weigh 403.2 mg freebase Form A and 125.1 mg oxalic acid dehydrate (˜1.2 eq.) into an HPLC vial.
Add 5 mL ACN to the vial. During the addition, the mixture formed a clear solution, the solution was seeded with little Oxalate Type A. A thick suspension formed immediately with seeding.
Magnetically stir at RT for 3 days.
Solids were isolated from the suspension by centrifugation. The wet sample showed obvious diffraction peaks of oxalic acid. The wet sample was slurry washed with ACN (1 mL, 5 min, 3 times) and the residual acid was removed.
The solids were vacuum-dried at RT overnight before characterization.

### Trial 3

Weigh 40.1 mg freebase Form A and 6.0 mg oxalic acid dehydrate (0.55 eq.) into an HPLC vial.
Add 0.5 mL ACN to the vial. Magnetically stir at RT for 3 days.
Solids were isolated from the suspension by centrifugation and conformed to Oxalate Type A.
IC/HPLC test revealed that the molar ratio of ion to freebase was 0.5.

### Trial 4

Weigh 40.1 mg freebase Form A and 23.6 mg oxalic acid dehydrate (2.2 eq.) into an HPLC vial.
Add 0.5 mL ACN to the vial. Magnetically stir at RT for 3 days.
Solids were isolated from the suspension by centrifugation. The wet sample showed obvious diffraction peaks of oxalic acid. The wet sample was slurry washed with ACN (1 mL, 5 min, 3 times) and the residual acid was removed.
IC/HPLC test revealed that the molar ratio of ion to freebase was 0.5.

### Trial 5

Weigh 40.1 mg freebase Form A and 10.7 mg oxalic acid dehydrate (1 eq.) into an HPLC vial.
Add 0.5 mL ACN to the vial. During the addition, the mixture formed a clear solution, which converted to suspension in seconds.
Magnetically stir at RT for 3 days.
Isolate the solids from the suspension by centrifugation and vacuum dry at RT overnight before characterization.

### Trial 6

Weigh 40.1 mg freebase Form A and 10.9 mg oxalic acid dehydrate (1 eq.) into an HPLC vial.
Add 0.5 mL Isopropyl Acetate (IPAc) to the vial to form a suspension.
Magnetically stir at RT for 3 days.
Isolate the solids from the suspension by centrifugation and vacuum dry at RT overnight before characterization.

### Trial 7

1. Weigh 39.9 mg freebase Form A and 10.7 mg oxalic acid dehydrate (1 eq.) into an HPLC vial.
2. Add 0.5 mL 2-Methyltetrahydrofuran (2-MeTHF) to the vial. During the addition, the mixture formed a clear solution, which converted to suspension in seconds.
3. Magnetically stir at RT for 3 days.
4. Isolate the solids from the suspension by centrifugation and vacuum dry at RT overnight before characterization.

### Trial 8

Weigh 15.3 mg amorphous oxalate (obtained by grinding of 99.8 mg freebase Type A and 26.6 mg (˜1 eq.) oxalic acid dihydrate in 3.75 mL IPAc) into an HPLC vial.
Add 0.2 mL ACN to the vial.
Allow for a one-week temperature cycling (One cycle was to heat to 50° C. at a rate of 4.5° C./min, isothermal for about 2 hrs, cool to 5° C. at a rate of 0.1° C./min, isothermal for about 2 hrs).
Isolate the solids from the suspension by centrifugation and vacuum dry at RT overnight before characterization.

### Trial 9

Weigh 14.7 mg amorphous oxalate (obtained by grinding of 99.8 mg freebase Type A and 26.6 mg (˜1 eq.) oxalic acid dihydrate in 3.75 mL IPAc) into an HPLC vial.
Add 0.2 mL IPAc to the vial.
Allow for a one-week temperature cycling (One cycle was to heat to 50° C. at a rate of 4.5° C./min, isothermal for about 2 hrs, cool to 5° C. at a rate of 0.1° C./min, isothermal for about 2 hrs).
Isolate the solids from the suspension by centrifugation and vacuum dry at RT overnight before characterization.

### Trial 10

Weigh 16.7 mg amorphous oxalate (obtained by grinding of 99.8 mg freebase Type A and 26.6 mg (˜1 eq.) oxalic acid dihydrate in 3.75 mL IPAc) into an HPLC vial.
Add 0.2 mL 2-MeTHF to the vial.
Allow for a one-week temperature cycling (One cycle was to heat to 50° C. at a rate of 4.5° C./min, isothermal for about 2 hrs, cool to 5° C. at a rate of 0.1° C./min, isothermal for about 2 hrs).
Isolate the solids from the suspension by centrifugation and vacuum dry at RT overnight before characterization.

### Polymorph Screening of Oxalate Type A

TABLE 6
Summary of Polymorph Screening in Oxalate Type A

| Experiment ID | Solvent, v:v | Results (7-days) |
|---|---|---|
| 1 | EtOH | Oxalate Type A |
| 2 | IPA | Oxalate Type A |
| 3 | 1-BuOH | Oxalate Type A |
| 4 | Butyl acetate | Oxalate Type A |
| 5 | 1-Propanol | Oxalate Type A |
| 6* | THF/H₂O, 1:1 | One peak |
| 7 | MeOAc | Oxalate Type A |
| 8 | MeOH/MTBE, 1:2 | Oxalate Type A + peak |
| 9* | 1,4-Dioxane/H₂O, 2:1 | One peak |
| 10 | CHCl₃/n-heptane, 1:1 | Oxalate Type A |
| 11 | DCM/toluene, 1:2 | Oxalate Type A |
| 12 | EtOAc | Oxalate Type A |
| 13 | Anisole | Oxalate Type A |
| 14 | CPME | Oxalate Type A |
| 15 | DMSO/H₂O, 1:4 | Oxalate Type A + freebase |
| | | Type A + peak |
| 16 | ACN/H₂O, 989:11, a_{w}~0.2 | Oxalate Type A |
| 17 | ACN/H₂O, 978:22, a_{w}~0.4 | Oxalate Type A |
| 18 | ACN/H₂O, 959:41, a_{w}~0.6 | Oxalate Type A |
| 19* | ACN/H₂O, 925:75, a_{w}~0.8 | Oxalate Type A (shift, wet) |
| | | Oxalate Type A (dry) |
| 20 | EtOAc saturated by water | Oxalate Type A |

### Solubility

Table 7 and Table 8 provide summaries of solubility experiment in water at 37° C. and 25° C., respectively.

TABLE 7
Summary of Solubility Experiment in Water (37° C.)

| | 1 hr | | | 2 hrs | | | 4 hrs | | | 24 hrs | | |
|---|---|---|---|---|---|---|---|---|---|---|---|---|
| Material | S | pH | FC | S | pH | FC | S | pH | FC | S | pH | FC |
| Form 1 | 0.090 | 3.22 | Gel | 0.088 | 2.97 | Gel | 0.073 | 3.01 | Gel | 0.082 | 2.74 | Gel |
| Freebase | 0.055 | 8.76 | No | 0.057 | 8.77 | No | 0.059 | 8.63 | No | 0.056 | 7.67 | No |
| Form A | | | | | | | | | | | | |
| Freebase | 0.089 | 8.78 | Gel | 0.089 | 8.77 | Gel | 0.084 | 8.60 | Gel | 0.078 | 7.74 | Gel |
| Amorphous | | | | | | | | | | | | |

S: solubility, mg/mL; FC: solid form change.
Solid loading: 2 mg/mL.

TABLE 8
Summary of Solubility Experiment in Water (25° C.)

| | 1 hr | | | 2 hrs | | | 4 hrs | | | 24 hrs | | |
|---|---|---|---|---|---|---|---|---|---|---|---|---|
| Material | S | pH | FC | S | pH | FC | S | pH | FC | S | pH | FC |
| Form 1 | 0.075 | 3.36 | No | 0.078 | 3.09 | No | 0.077 | 3.01 | No | 0.075 | 2.76 | No |
| Freebase | 0.044 | 8.66 | No | 0.046 | 8.66 | No | 0.040 | 8.57 | No | 0.047 | 7.85 | No |
| Form A | | | | | | | | | | | | |
| Freebase | 0.074 | 8.55 | No | 0.081 | 8.57 | No | 0.078 | 8.51 | No | 0.072 | 7.68 | No |
| Amorphous | | | | | | | | | | | | |

S: solubility, mg/mL; FC: solid form change.
Solid loading: 2 mg/mL.

### Cocrystal Solid Form 1

Form 1 was prepared with the method used in Trial 2 when oxalic acid dehydrate was used and ACN was used as solvent. Form 1 is a crystalline solid form of Compound 1 and oxalic acid.

FIG. 1 provides an XRPD pattern of Form 1. A list of X-Ray Diffraction Peaks for Form 1 is provided below in Table 9.

TABLE 9
X-Ray Diffraction Peaks for Form 1

| Pos. [°2θ] | d-spacing [Å] | Rel. Int. [%] |
|---|---|---|
| 4.1183 | 21.46 | 23.69 |
| 8.1662 | 10.83 | 85.91 |
| 8.5601 | 10.33 | 31.86 |
| 10.3171 | 8.57 | 4.75 |
| 11.6459 | 7.60 | 100.00 |
| 12.2378 | 7.23 | 12.93 |
| 15.6723 | 5.65 | 7.64 |
| 16.5254 | 5.36 | 62.43 |
| 16.8462 | 5.26 | 56.77 |
| 17.7079 | 5.01 | 34.16 |
| 18.2251 | 4.87 | 33.87 |
| 18.5356 | 4.79 | 74.62 |
| 18.7958 | 4.72 | 38.59 |
| 19.5604 | 4.54 | 66.06 |
| 19.9261 | 4.46 | 37.28 |
| 20.4936 | 4.33 | 62.99 |
| 20.6885 | 4.29 | 72.02 |
| 21.2597 | 4.18 | 18.28 |
| 21.6081 | 4.11 | 85.08 |
| 21.9631 | 4.05 | 4.50 |
| 22.8391 | 3.89 | 44.40 |
| 23.0176 | 3.86 | 39.86 |
| 23.2905 | 3.82 | 24.72 |
| 23.7338 | 3.75 | 12.06 |
| 24.3513 | 3.66 | 9.90 |
| 25.1246 | 3.54 | 13.42 |
| 25.3663 | 3.51 | 5.62 |
| 25.6280 | 3.48 | 12.26 |
| 25.7911 | 3.45 | 12.47 |
| 26.0389 | 3.42 | 5.04 |
| 26.3567 | 3.38 | 4.28 |
| 27.1092 | 3.29 | 8.60 |
| 27.5212 | 3.24 | 10.89 |
| 27.9227 | 3.20 | 3.63 |
| 28.2391 | 3.16 | 5.16 |
| 28.9005 | 3.09 | 11.95 |
| 29.5992 | 3.02 | 2.68 |
| 29.9361 | 2.98 | 5.18 |
| 30.2906 | 2.95 | 15.71 |
| 30.7923 | 2.90 | 5.80 |
| 31.4198 | 2.85 | 3.41 |
| 33.0259 | 2.71 | 3.52 |
| 33.3489 | 2.69 | 5.91 |
| 33.9865 | 2.64 | 10.14 |
| 35.1273 | 2.55 | 3.05 |
| 35.8528 | 2.50 | 1.80 |
| 37.2248 | 2.42 | 3.17 |
| 38.7823 | 2.32 | 2.06 |
| 39.5788 | 2.28 | 0.99 |

FIG. 2 and FIG. 3 provide TGA data and DSC data of Form 1, respectively. A mass loss of 0.70% between about 17.6° C. and about 130° C. was observed in FIG. 2. According to the DSC sig data nal in FIG. 3, an endothermic melt event was observed with an onset temperature of 159.1° C. and a peak temperature at 160.4° C.

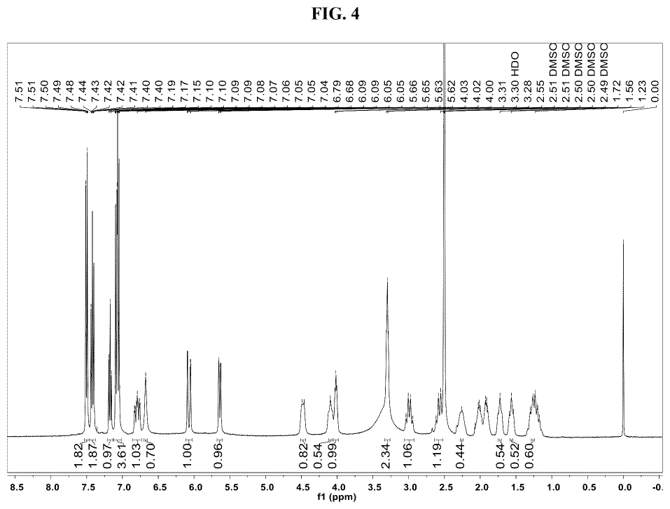

FIG. 4 provides a ¹H NMR of Form 1.

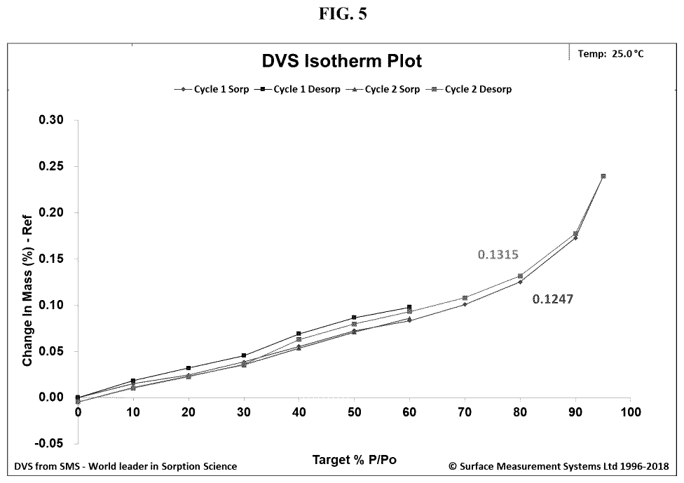

FIG. 5 provides a DVS result showing that water uptake of Form 1 at 25° C./80% RH was lower than 0.2%, indicating it was non hygroscopic.

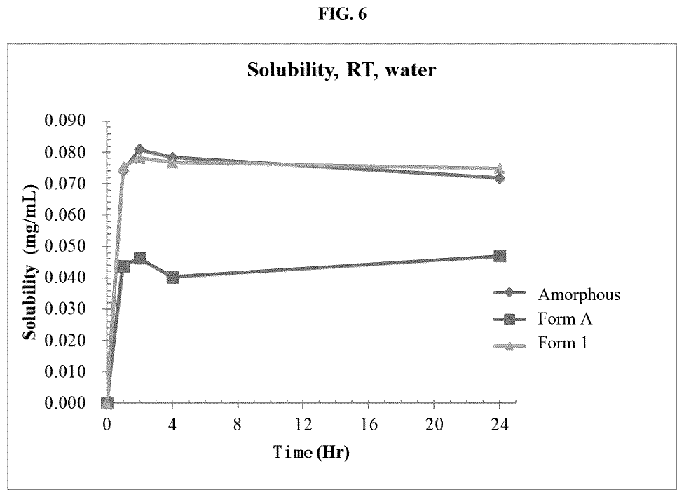

FIG. 6 provides a solutability profile of Form A of Compound 1, and amorphous form of Compound 1 at room temperature.

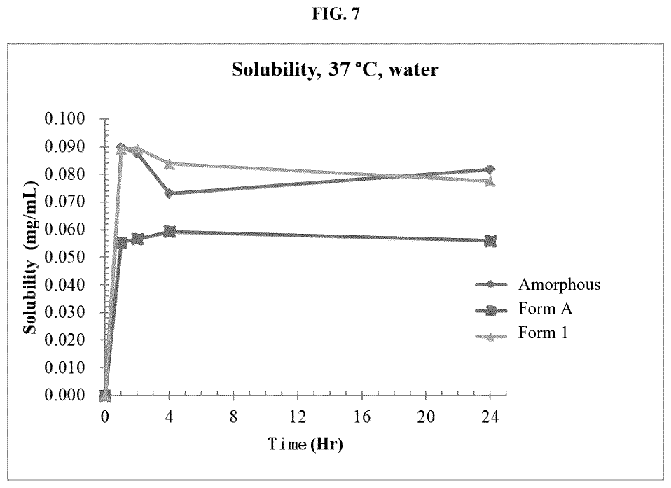

FIG. 7 provides a solutability profile of Form A of Compound 1, and amorphous form of Compound 1 at 37° C.

Two samples were prepared for stoichiometry test. Each sample was tested by HPLC for Compound 1 concentration and tested by IC for oxalic acid concentration. The HPLC and IC results showed the molar ration of oxalic acid to Compound 1 was 0.5.

At least the following methods may be used to determine whether Form 1 of Compound 1 is a cocrystal and whether Form 1 of Compound 1 is a salt:
(1) Single crystal X-ray diffraction which is the most common way to distinguish a cocrystal from a salt. Standard experimental method, procedure, and parameter may be found in Cullity, B. D., & Stock, S. R. (2001). Elements of X-Ray Diffraction (3rd ed.). Pearson; and Pecharsky, V., & Zavalij, P. (2008). Fundamentals of Powder Diffraction and Structural Characterization of Materials, Second Edition (2nd ed. 2009 ed.). Springer.
(2) Solid State NMR. Standard experimental method, procedure, and parameter may be found in Apperley, D. C., Harris, R. K., & Hodgkinson, P. (2012c). Solid-state NMR. Amsterdam University Press.
(3) X-ray photoelectron spectroscopy. Standard experimental method, procedure, and parameter may be found in der Heide, V. P. (2011). X-ray Photoelectron Spectroscopy: An introduction to Principles and Practices (1st ed.). Wiley.
   Solid state NMR and X-ray photoelectron spectroscopy do not require a single crystal.

The embodiments disclosed herein are not to be limited in scope by the specific embodiments disclosed in the examples which are intended as illustrations of a few aspects of the disclosed embodiments and any embodiments that are functionally equivalent are encompassed by the present disclosure. Indeed, various modifications of the embodiments disclosed herein are in addition to those shown and described herein will become apparent to those skilled in the art and are intended to fall within the scope of the appended claims.

A number of references have been cited, the disclosures of which are incorporated herein by reference in their entirety.

1. A solid form comprising (a) (S)-7-(1-acryloylpiperidin-4-yl)-2-(4-phenoxyphenyl)-4,5,6,7-tetrahydropyrazolo[1,5-a]pyrimidine-3-carboxamide; and (b) oxalic acid.

2. The solid form of claim 1, which is substantially crystalline.

3. The solid form of claim 2, which is substantially a cocrystal.

4. The solid form of claim 2, which is substantially a salt.

5. The solid form of claim 2, which is a crystal form comprising Compound 1: 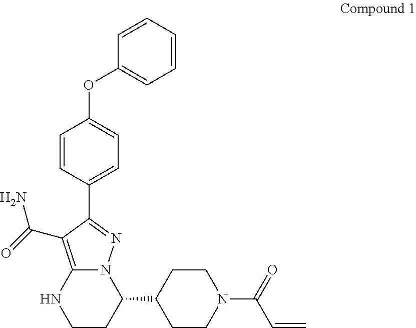 [SMILES: C=CC(=O)N1CCC([C@@H]2CCNc3c(C(N)=O)c(-c4ccc(Oc5ccccc5)cc4)nn32)CC1]
which has an X-ray powder diffraction pattern comprising peaks at approximately 8.2, 11.7, and 21.6°2θ.

6. The crystal form of claim 5 which has an X-ray powder diffraction pattern further comprising peaks at approximately 8.6, 18.5, and 19.6°2θ.

7. The crystal form of claim 5 which has a thermogravimetric analysis thermogram comprising a total mass loss of approximately 0.7% of the total mass of the crystal form when heated from about 17.6° C. to about 130° C.

8. The crystal form of claim 5 which has a differential scanning calorimetry thermogram comprising an endothermic event with an onset temperature at approximately 159.1° C. when heated from about 50° C. to about 200° C.

9. The crystal form of claim 5 which is an oxalic acid co-crystal form.

10. The crystal form of claim 5 which is an oxalic acid salt.

11. The crystal form of claim 5, wherein the molar ratio of oxalic acid to Compound 1 is about 0.5.

12. The crystal form of claim 5 which is substantially pure.

13. A method for treating or preventing a B-cell proliferative disease, or a condition treatable or preventable by inhibition of a kinase pathway, comprising administering an effective amount of a solid form of claim 1 to a subject in need thereof.

14. The method of claim 13, wherein the kinase pathway is the BTK pathway.

15. A method for achieving a Response Evaluation Criteria in Solid Tumors (RECIST 1.1) of complete response, partial response or stable disease in a subject comprising administering an effective amount of a solid form of claim 1 to a subject having a solid tumor.
